(19) United States
(12) Patent Application Publication
(10) Pub. No.: US 2005/0203456 A1
(43) Pub. Date: Sep. 15, 2005

(54) Retrievable gauze pad

(76) Inventor: Dan W. Dansie, Payson, UT (US)

Correspondence Address: Dan Dansie, 380 West Utah Ave., Payson, UT, 84651, US

(21) Appl. No.: 10/797,233

(22) Filed: Mar. 10, 2004

(51) Int. Cl. A61F 13/00

(52) U.S. Cl. 602/42; 604/358

(57) ABSTRACT

An apparatus used during oral surgery having two major components: an absorbent gauze pad approved for use during oral surgery, and a means for retrieving the absorbent gauze pad attached to the absorbent gauze pad. The resulting retrievable gauze pad allows a patient having undergone oral surgery, or another person, to easily remove the retrievable gauze pad, especially if the patient accidentally aspirates the retrievable gauze pad.

### BACKGROUND

1. The Field of the Invention

The present invention relates to an apparatus used to absorb blood and other fluids during numerous types of oral surgery. The present invention also relates to dramatically increasing the patient's safety during oral surgery.
2. Brief Description of the Art

Oral surgery of all forms presents a certain amount of risks and danger. A dentist performing oral surgery will use a number of gauze pads, sponges or combination of devices to absorb blood and other fluids naturally present in the oral cavity during surgery. The absorbent materials are available in a variety of designs and sizes.

The general purpose for these absorbent materials is to prevent blood and saliva from seeping into the naso-pharynx, trachea and esophagus. Large pieces of absorbent material may restrict the airway. Small pieces of absorbent material may become dislodged and easily swallowed or aspirated. The absorbent material is usually packed into numerous areas of the oral cavity, and may be pushed deep into the naso-pharynx.

Generally, the patient is told by the dentist to leave the absorbent material in their oral cavity for a relatively extended time after oral surgery is complete. The vast majority of dentists currently practicing do not attach anything that would assist a patient in removing the absorbent material packed into the patient's oral cavity. There are certain risks associated with this procedure.

It is well understood by those skilled in the art that the oral cavity is greatly anesthetized during oral surgery. The anesthesia causes paresthesia of the mandible, or numbness or the hard palate, soft palate and root of the tongue. This resulting paresthesia suppresses the gag reflex, which reflex is located in the root of the tongue. The suppression of the gag reflex, and the paresthesia of the mandible, generally lasts six to eight hours after completion of oral surgery, but may last as long as twelve hours. The use of anesthesia may also cause a general, mental incoherence.

While the patient is still anesthetized, and after the patient has left the dentist's office, it is not uncommon for the patient to swallow a piece of the absorbent material. The paresthesia of the mandible and the resulting suppression of the gag reflex allows a piece of absorbent material to work down, or travel, deep into the naso-pharynx without the knowledge of the patient. The patient simply cannot feel the absorbent material, or a piece thereof, during this period of recuperation.

When a piece of absorbent material is swallowed by a patient, the paresthesia of the mandible prevents the patient from feeling the piece of absorbent material as it enters the patient's esophagus. The paresthesia does not allow the patient to control the voluntary muscles controlling the top of the esophagus. As the piece of absorbent material travels down the esophagus, involuntary muscles control the swallowing of the absorbent material. The patient will begin to feel the absorbent material as it travels further down the esophagus. While swallowing a piece of absorbent material may result in some discomfort for the patient, it is not considered dangerous for the patient.

The fact that dentists do not consider swallowing a piece of absorbent material dangerous contributes greatly to the fact that the vast majority of dentists do not attach retrieval aids to the absorbent material used during oral surgery.

While it is rare, patients with paresthesia of the mandible, and the resulting suppression of the gag reflex, have aspirated pieces of absorbent material left in the patient's oral cavity after oral surgery. Fatalities have occurred because a patient accidentally aspirated a piece of absorbent material. A patient who aspirates a piece of absorbent material has virtually no chance of receiving medical attention in time to save their life.

A piece of absorbent material will travel deep into the patient's naso-pharynx, similar to when a patient swallows a piece of absorbent material, but the epiglottis will not close the trachea, or airway, and the piece of absorbent material will travel toward the lungs. The piece of absorbent material will become lodged in the trachea at the first branching of the trachea, known as the furcation of the lungs.

Once a piece of absorbent material is lodged at the furcation of the lungs, the patient cannot breathe. Tragically, death is usually imminent because this chain of events has happened so quickly that the patient is probably not near any type of emergency facility.

Despite the dire consequences, the possibility of a patient aspirating a piece of absorbent material during the period of recuperation immediately following oral surgery is considered negligible by most dentists. Therefore, the vast majority of dentists performing oral surgery do not attach any type of retrieval aid to absorbent material used during oral surgery.

Those dentists that do attach a retrieval aid to absorbent material used during oral surgery generally tie a length of dental floss to the absorbent material. The dental floss helps ensure no individual piece of absorbent material is lost, and helps the patient remove all absorbent material remaining after oral surgery. However, the absorbent material becomes distorted and misshapen after the dental floss is tied to it, and it is possible the absorbent material slip out of the dental floss.

### SUMMARY OF THE INVENTION

The current invention is amazingly simple. A length of string is attached to a piece of gauze by sewing the string into the gauze. Generally, the gauze is non-woven, rayon/polyester formed gauze (8). This gauze is a specific type of gauze frequently used by dentists and approved by appropriate dental regulatory authorities. The string is usually durable, nylon string.

Sewing the string into the gauze ensures the entire piece of gauze will be removed when the string is used to remove the gauze. Also, sewing allows the string to be attached to the gauze without distorting or misshaping the gauze.

The fact that this invention will be readily available and easy to use may very well lead to this invention being designated as the standard of care for oral surgery. Currently, the vast majority of dentists do not use absorbent material having an aid for retrieval attached thereto. The most common reasons for this are: dentists do not consider a patient aspirating a piece of absorbent material very likely; dentists do not consider a patient swallowing a piece of absorbent material to be dangerous; and dentists do not want to take the time necessary to attach their own means of aiding retrieval to every piece of absorbent material used during oral surgery.

Although non-woven, rayon/polyester formed gauze (8) and nylon string are designated here as the principal components of the apparatus, those skilled in the art will recognize that any means aiding retrieval may be attached to any absorbent material suitable for use in oral surgery.

### BRIEF DESCRIPTION OF THE DRAWINGS

FIG. 1 is a perspective view of the retrievable gauze apparatus.

FIG. 2A is a perspective view of the first step in production of the retrievable gauze apparatus, the unfolded sheet of gauze.

FIG. 2B is a perspective view of the second step in production of the retrievable gauze apparatus, the first fold of the sheet of gauze.

FIG. 2C is a perspective view of the third step in production of the retrievable gauze apparatus, the second fold of the sheet of gauze.

FIG. 3 is a cross-sectional view of an individual showing the physical structures associated with use of the retrievable gauze pad.

### DETAILED DESCRIPTION OF THE PREFERRED EMBODIMENTS

It will be readily understood that the following detailed description of the embodiments of the retrievable gauze apparatus 10, and the steps for using the retrievable gauze apparatus 10, is not intended to limit the scope of the invention, as claimed, but is merely representative of the presently preferred embodiments of the invention.

1. Structure of the Retrievable Gauze Pad

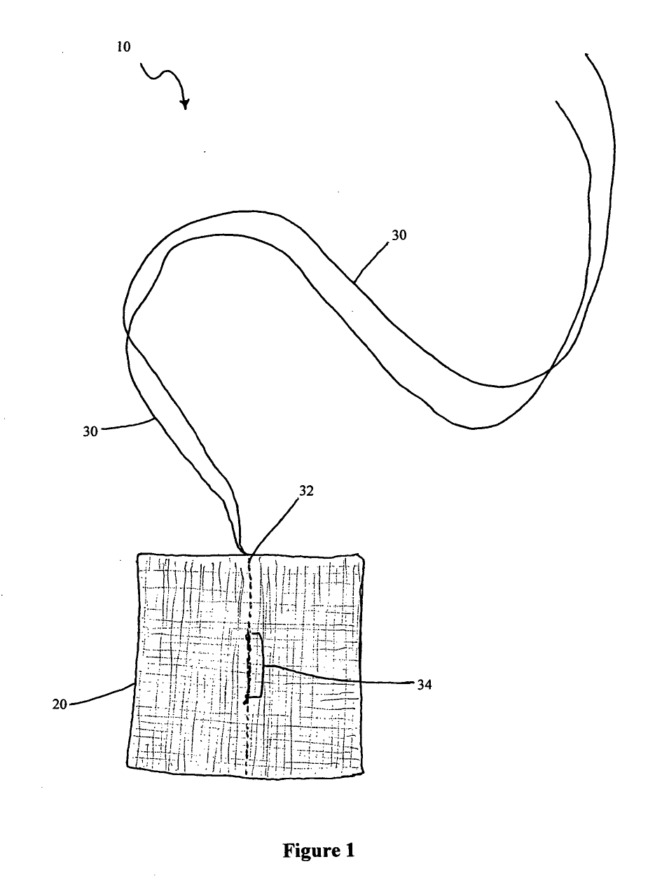

The structure of the apparatus 10 is very simple. As shown in FIG. 1, the apparatus 10 has two primary components: an absorbent pad 20 (or means for absorbing fluids commonly found during oral surgery) and string 30 (or means for retrieving the means for absorbing fluids commonly found during oral surgery).

The absorbent pad 20, or means for absorbing fluids, is generally non-woven, nylon/polyester gauze (8). This is a specific type of gauze commonly used by dentists and approved for use by dental regulatory authorities. The retrievable gauze pad 10 is designed to be used during oral surgery, therefore, an absorbent pad 20 needs to be composed of absorbent material approved for such use by proper authorities.

The absorbent pad 20 can be any suitable size or shape. Generally, the absorbent pad 20 is a piece of gauze four (4) inches wide by four (4) inches high which is folded in half twice, resulting in an absorbent pad 20 two (2) inches wide by two (2) inches high. However, a square, rectangular or circular piece of absorbent material would also be suitable. Any size convenient for use during oral surgery is also acceptable.

The string 30, or means for retrieving, is generally durable, nylon string. More than one string 30 may be attached to the absorbent pad 20. The string 30 can be any material suitable for use during oral surgery. The color of the string 30 is generally chosen so as to contrast with the patient and associated fluids present during oral surgery, thus making the string easy to locate and distinguish. As previously mentioned, dental floss could be used as the string 30. As will be discussed in greater detail, the string 30 must be at least fourteen (14) inches in length.

The string 30 is generally attached to the absorbent pad 20 by sewing the string 30 into the absorbent pad 20. As a result of the sewing process, stitching 32 can be seen vertically across the surface of the absorbent pad 20. A sewing process called backstitching creates an area along the stitching 32, designated as the backstitch 34, located near the middle of the stitching 32. Backstitching helps ensure the string 30 will not become separated from the absorbent pad 20.

As will be recognized by those skilled in the art, any suitable process for attaching the sting 30 to the absorbent pad 20 may be used. Sewing and weaving would be examples of means for attaching the string 30 to the absorbent pad 20.

2. Production of the Retrievable Gauze Pad

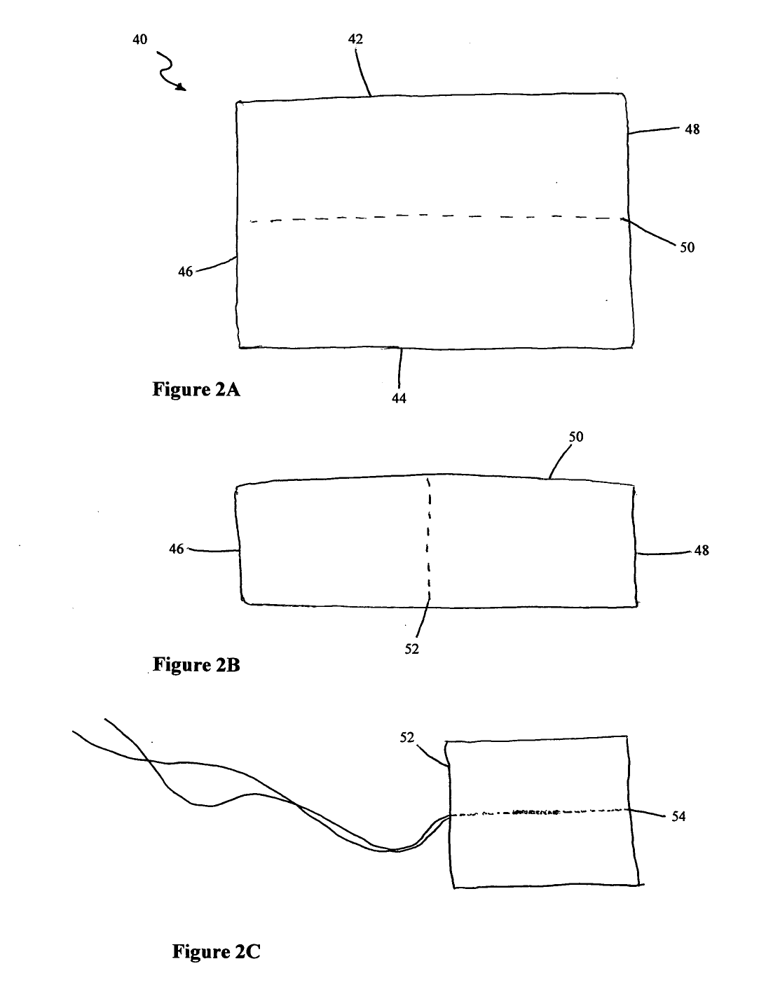

The first step in producing a retrievable gauze pad 10 is to provide an unfolded sheet of gauze 40, as shown in FIG. 2A. The unfolded sheet of gauze 40 will have a top edge 42 and a bottom edge, each approximately four (4) inches. The unfolded sheet of gauze 40 will have a left edge 46 and a right edge 48, each approximately four (4) inches.

As shown in FIG. 2B, the second step in production is to fold the unfolded sheet of gauze 40 basically in half. The first fold 50 may be performed by aligning the top edge 42 with the bottom edge 44, resulting in a pad of gauze approximately four (4) inches wide and two (2) inches high.

As shown in FIG. 2C, the third step in production is to fold the pad of gauze a second time, basically in half. The second fold 52 may be performed by aligning the left edge 46 with the right edge 48, resulting in a pad of gauze approximately two (2) inches wide and two (2) inches high. All the folding may be done by hand or by machine.

The string 30 is attached to the folded pad by sewing the string 30 into the folded absorbent pad 20 along the median 54. The resulting stitching 32 travels vertically along the surface of the absorbent pad 20. The technique of backstitching is used to ensure the string 30 does not become separated from the absorbent pad 20, and a backstitch 34 is seen in the approximate middle of the stitching 32. Additional stitching 32 can be used as desired to more completely secure the string 30 to the absorbent pad 20.

The retrievable gauze pad 10 may be mass produced using different techniques. For example, four rolls of two inch wide gauze may be arranged so the four rolls supply each of the four layers of gauze used in a retrievable gauze pad 10. The two inch wide gauze may be extended and the stitching 32, including the backstitch 34, sewn into the four layers of two inch wide gauze at two inch intervals. Then the layers of gauze will be cut between the intervals of stitching 32 resulting in retrievable gauze pads 10 approximately two inches wide by two inches high.

Generally the retrievable gauze pad 10 is packaged in a manner that allows the user to readily sterilize the retrievable gauze pad 10 prior to use. However, it is possible to sterilize the retrievable gauze pad 10 after production and then deliver the retrievable gauze pad 10 to the user.

3. Use of the Retrievable Gauze Pad

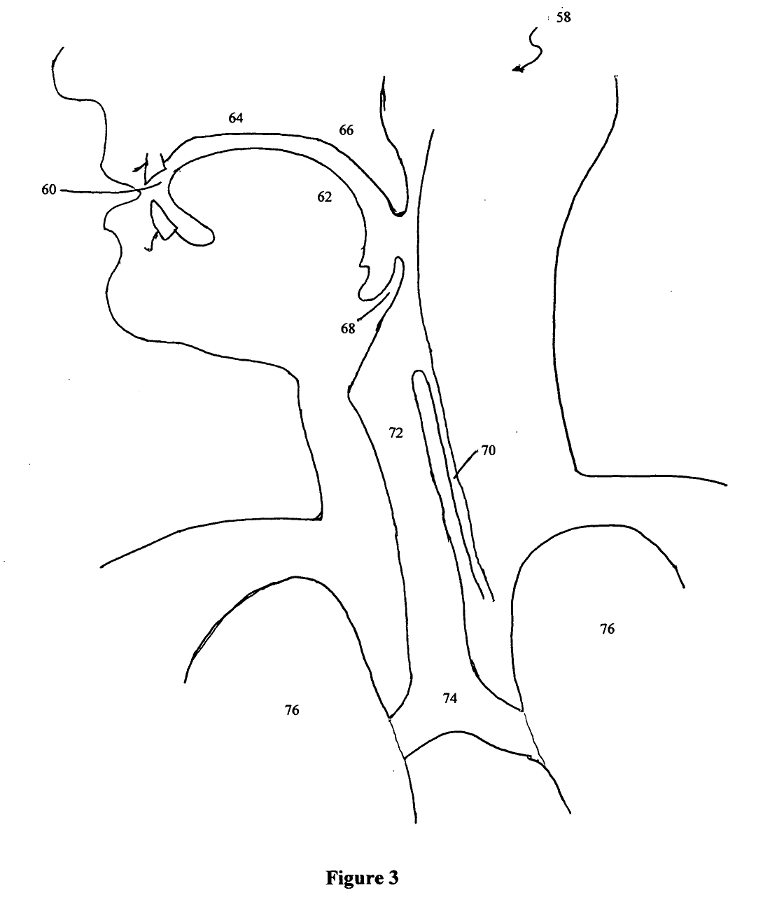

FIG. 3 is intended to illustrate use of the retrievable gauze pads 10. During oral surgery, a patient 58 may have numerous retrievable gauze pads 10 placed in their mouth 60. The retrievable gauze pads 10 may be placed in numerous positions in the mouth 60, including without limitation, between the cheek and gums, between the gums and tongue, over teeth, and near the root of the tongue 62.

The patient 58 may experience various types of anesthesia prior to and during oral surgery. A topical anesthesia may be used to prepare a patient 58 for anesthesia injections. The use of anesthesia may result in a condition known as temporary paresthesia of the mandible. Paresthesia of the mandible is characterized by numbness of the hard palate 64, soft palate 66, and root of the tongue 62. The gag reflex is located in the root of the tongue 62 and the soft palate 66. Paresthesia of the mandible causes suppression of the gag reflex.

A dentist may perform a palatine block of the palatine nerve. When the palatine nerve is numbed, paresthesia of the hard palate 64 and soft palate 66 results. A dentist may also perform a mandibular block of the mandibular nerve, also known as the inferior alveolar nerve. When the mandibular nerve is numbed, paresthesia of the root of the tongue 62 results. During the mandibular block, the lingual nerve is also numbed resulting in the forward part of the tongue being numbed as well.

As discussed earlier, a patient 58 experiencing oral surgery may have numerous retrievable gauze pads 10 placed in their mouth 60 during oral surgery. The patient 58 may need to leave the retrievable gauze pads 10 in their mouth for several hours after completion of the oral surgery. The retrievable gauze pads 10 may travel, or be forced, into the back of the mouth 60 and swallowed or aspirated by the patient 58. All of this may occur unnoticed by the patient 58 due to the lingering effects of the paresthesia of the mandible.

When a patient 58 swallows a retrievable gauze pad 10, the absorbent pad 20 may travel to the back of the mouth 60. The epiglottis 68 may close the opening to the trachea 72, forcing the absorbent pad 20 into the esophagus 70. As the absorbent pad 20 begins to travel down the esophagus 70, involuntary muscles will eventually control the progress of the absorbent pad 20 down the esophagus 70. As the absorbent pad 20 enters and travels down the esophagus 70, the patient 58 may feel the absorbent pad being swallowed. The patient may be able to pull the string 30 attached to the absorbent pad 20, thereby removing the absorbent pad 20 from the esophagus 70. The patient 58 may also be able to choke or cough and hold the absorbent pad 20 in place long enough to locate the string 30 attached to the absorbent pad 20, and then remove the absorbent pad 20 from the esophagus 70.

If the patient 58 swallows the retrievable gauze pad 10, while an uncomfortable experience, generally, no physical danger will result. However, the retrievable gauze pad 10 may become lodged in the digestive tract, causing serious physical danger. For example, the circular esophageal muscle may spasm causing the retrievable gauze pad 10 to be lodged in the esophagus 70, especially in elderly patients.

When the patient 58 aspirates a retrievable gauze pad 10, the absorbent pad 20 may travel to the back of the mouth 60. The epiglottis 68 may remain over the opening to the esophagus 70, allowing the absorbent pad 20 into the trachea 72. As the absorbent pad 20 begins to travel down the trachea 72 toward the lungs 76, it reaches the first branching of the trachea 72, the furcation 74 of the lungs 76. Generally, the furcation 74 of the lungs 76 is located approximately twelve (12) inches from the opening of the mouth 60. As the absorbent pad 20 becomes lodged in the trachea 72 at the furcation 74 of the lungs 76, the end of the string 30 will remain outside the mouth 60, allowing the patient 58 to pull the string 30 attached to the absorbent pad 20, thereby removing the absorbent pad 20 from the furcation 74 of the lungs 76. Removing the absorbent pad 20 from the furcation 74 of the lungs 76 will open the trachea 72 and allow the patient 58 to breathe.

If an absorbent pad 20 is aspirated by a patient 58, that patient 58 will be unable to breathe, and the patient will likely die in a short amount of time. Obviously, the removal of an absorbent pad 20 lodged in the trachea 72 at the furcation 74 of the lungs 76 will likely save the life of the patient 58.

What is claimed and desired to be secured by United States letters patent is:

1. An apparatus for use during oral surgery comprising:
an absorbent pad; and
a string attached to said absorbent pad, said string being at least fourteen (14) inches in length.

2. An apparatus as in claim 1 wherein said absorbent pad is non-woven, rayon/polyester formed gauze (8).

3. An apparatus as in claim 1 wherein said string is nylon.

4. An apparatus as in claim 3 further comprising a second string attached to said absorbent pad, said second string being at least fourteen (14) inches in length.

5. An apparatus as in claim 1 wherein said string is colored so as to contrast with a person's oral cavity and said person's bodily fluids.

6. An apparatus as in claim 1 wherein said apparatus is sterilized.

7. An apparatus for use during oral surgery comprising:
means for absorbing fluids commonly found during oral surgery; and
means for retrieving said means for absorbing fluids, attached to said means for absorbing fluids, and being at least fourteen (14) inches in length.

8. An apparatus as in claim 7 wherein said means for absorbing fluids is a non-woven, rayon/polyester formed gauze (8).

9. An apparatus as in claim 7 wherein said means for removing is at least one nylon string.

10. An apparatus as in claim 7 wherein said apparatus is sterilized.

11. A method for making an apparatus for use during oral surgery comprising:
providing a sheet of gauze being approximately four (4) inches high by four (4) inches wide;
performing a first fold of said sheet of gauze, said first fold resulting in said sheet of gauze being approximately two (2) inches high by four (4) inches wide;
performing a second fold of said sheet of gauze, said second fold resulting in said sheet of gauze being approximately two (2) inches high by two (2) inches wide;
providing nylon string; and
sewing said nylon string along a median of said sheet of gauze so as to leave at least fourteen (14) inches of string extending beyond said sheet of gauze.

12. The method of claim 11 further comprising:
using the technique of backstitching to ensure secure attachment of said nylon string during said sewing of said nylon string.
